(19) United States
(12) Patent Application Publication
(10) Pub. No.: US 2008/0254150 A1
(43) Pub. Date: Oct. 16, 2008

(54) Management of dermatitic symptoms of mammalian integument with emollient disinfectant formulations

(76) Inventors: Lawrence A. Rheins, Glendale, AZ (US); John C. Hill, Mesa, AZ (US); David Ashley, Phoenix, AZ (US); James S. Brown, Gilbert, AZ (US); John Reinhardt, Riverside, CA (US); James H. Brown, Phoenix, AZ (US)

Correspondence Address: International Flora Technologies, Ltd.;C/O Intellevate LLC, P.O. Box 52050, Minneapolis, MN, 52050, US

(21) Appl. No.: 12/080,070

(22) Filed: Mar. 31, 2008

Related U.S. Application Data
(63) Continuation-in-part of application No. 10/611,775, filed on Jun. 30, 2003.
Application No. 10/611,775 is a continuation-in-part of application No. 09/478,071, filed on Jan. 3, 2000.
(60) Provisional application No. 60/920,604, filed on Mar. 29, 2007.

(51) Int. Cl. A61K 36/36; A61K 31/045; A61K 31/047; A61P 17/00

(52) U.S. Cl. 424/725; 514/724; 514/738

(57) ABSTRACT

Botanically-sourced and botanically-derived emollient sanitation compositions for topical use are disclosed. Representative compositions generally aid reconstitution of the lipid profile of the stratum corneum by providing botanical lipids and/or lipid-derivatives that resemble human sebum—these components being ordinarily diminished with the use of conventional hand sanitizer products. Disclosed features and specifications may be variously controlled, adapted or optionally modified to realize, for example, improved hand sanitizer formulations. Representative embodiments of the present invention generally provide anti-microbial compositions blended with botanically sourced lipids and/or lipid-derivatives to control or otherwise improve dermatitic symptoms associated with frequent use of conventional hand sanitizer products.

### RELATED APPLICATIONS

This application claims the benefit of U.S. Provisional Patent Application Ser. No. 60/920,604 filed in the United States Patent and Trademark Office on Mar. 29, 2007 by Lawrence A. Rheins, John C. Hill, Grace Hastings, James H. Brown, and John Reinhardt, and is a continuation-in-part of U.S. patent application Ser. No. 10/611,775 filed in the United States Patent and Trademark office on Jun. 30, 2003 by John C. Hill and U.S. patent application Ser. No. 09/478,071 filed in the United States Patent and Trademark Office on Jan. 3, 2000 by James H. Brown, Lee Roy Copeland, Robert Kleiman, Sambasivarao Koritala, and Melanie K. Cummings.

### FIELD OF INVENTION

The present invention generally relates to emollient and sanitation compositions; and more particularly, representative embodiments of the present invention generally concern delivery of emollients in topically applied disinfectant formulations.

### BACKGROUND OF INVENTION

The spread of infectious disease due to inadequate hand hygiene is generally acknowledged by the scientific community and accepted by the public at large. As reported by the United States National Institute of Allergy and Infectious Diseases in 2006, the escalating incidence of nosocomial acquired infections by patients lead to approximately two million (2,000,000) hospital acquired infections per year and approximately ninety thousand (90,000) deaths in the United States alone, as compared to about thirteen thousand (13,000) deaths in 1992. This is especially disturbing due to the rapid development and spread of antibiotic-resistant bacteria, fungi, and parasites as well as antiviral, drug-resistant viruses. Antibiotic resistant strains of disease-causing bacteria, such as Staphylococcus aureus, are now commonly acquired in hospital settings due to close contact of patients who are more susceptible to infection and the extensive use of antibiotics, which generally provide selection pressure for these strains of bacteria. Consequently, people infected with these microbes are likely to have longer hospital stays and may require treatment with second- and third-choice antibiotics that may be less effective and more expensive.

Despite the knowledge that frequent hand washing is an effective preventative measure against the spread of disease-causing microbes, a significant level of healthcare worker non-compliance persists. Although most workers in the healthcare industry are regulated by policies requiring frequent hand washing and/or the use of liquid hand sanitizers, non-compliance with these policies has been reported to be between 45% and 70%. A prominent reason cited for non-compliance is the incidence of acute and chronic irritated skin and, to a lesser extent, contact allergic hand dermatitis due to repeated use of antibacterial soaps and the use of alcohol-based (either ethanol or isopropanol, 60%-95% wt/wt) hand sanitizers. The use of these sanitizers can be as high as fifty or more times during each work day.

A negative side effect of the use of conventional ethanol hand sanitizers, upon application to the skin, is that they generally operate to remove various surface lipids from the uppermost region of the skin known as the stratum corneum. These lipids typically function to maintain homeostatic balance of the skin. The chronic stripping of the lipid barrier usually results in xerosis, scaling, erythema, rough skin, and tight skin. More serious and painful side effects include inflammation, fissures, allergic contact dermatitis, and the harboring of transient pathogenic organisms that may cause infections. Common sensations associated with de-lipidization include itching, tingling, burning, stinging, and the like. Non-compliance that results from experiencing these types of side effects with the use of conventional hand sanitizers actually leads to further spread of diseases that hand-hygiene guidelines are promulgated with the intent of preventing.

As a mechanism for addressing adverse side effects, many individuals turn to moisturizers, corticosteroids, and the like; however, these mechanisms for replenishing moisture and/or combating dryness and other skin irritations are of limited efficacy when multiple hand cleansing cycles throughout the day are required. This is due to each cleansing cycle operating to remove the previously applied moisturizers as they sit on the uppermost surface of the skin—thereby reducing the exposure of the skin to the moisturizer and the moisturizer's overall effectiveness. Accordingly, there is a need for alternative sanitizer formulations to reduce the negative effects associated with frequent washing while maintaining effective disinfectant function.

### SUMMARY OF THE INVENTION

In a representative aspect, the present invention provides compositions and methods for providing botanically-sourced and/or botanically-derived topical emollient compositions with disinfectant properties to ameliorate dermatitic symptoms of mammalian integument. The sanitizing component of the composition may include an anti-microbial sanitizer. The emollient component of the composition may include botanical lipid materials (and/or their derivatives) selected to demonstrate properties at least partially analogous to mammalian sebum. The combination of sanitizing and emollient components of the resulting formulations may be employed to manage dermatitic symptoms and sanitize mammalian integument.

Advantages of the present invention will be set forth in the Detailed Description which follows and may be apparent in view of the Detailed Description or may be learned by practice of exemplary embodiments of the invention. Still other advantages of the invention may be realized by means of any of the instrumentalities, methods or combinations particularly pointed out in the claims.

### BRIEF DESCRIPTION OF THE DRAWINGS

Representative elements, operational features, applications and/or advantages of the present invention reside in the details of construction and operation as more fully hereafter depicted, described and claimed—reference being made to the accompanying drawings forming a part hereof, wherein like numerals refer to like parts throughout. Other elements, operational features, applications and/or advantages may become apparent in light of certain exemplary embodiments recited in the Detailed Description, wherein:

FIG. 1 illustrates clinical data relating to transepidermal water loss (TEWL) associated with use and non-use of an emollient sanitizing formulation in accordance with a representative embodiment of the present invention;

FIG. 2 illustrates clinical data relating to TEWL associated with use and non-use of an emollient sanitizing formulation in accordance with a representative embodiment of the present invention;

FIG. 3 is a photomicrographic representation of a cross-section of human skin tissue obtained via punch biopsy prior to application of an emollient sanitizing composition in accordance with a representative embodiment of the present invention; and

FIG. 4 is a photomicrographic representation of a cross-section of human skin tissue obtained via punch biopsy after fourteen (14) days of regular application (at least 8 times/day) of an emollient sanitizing composition in accordance with a representative embodiment of the present invention.

Elements in the Figures are illustrated for simplicity and clarity and have not necessarily been depicted to scale. For example, the dimensions of some of the elements in the Figures may be exaggerated relative to other elements to help improve understanding of various embodiments of the present invention. Furthermore, the terms “first”, “second”, and the like herein, if any, are used inter alia for distinguishing between similar elements and not necessarily for describing a sequential or chronological order.

### DETAILED DESCRIPTION OF EXEMPLARY EMBODIMENTS

The following representative descriptions of the present invention generally relate to exemplary embodiments and the inventors' conception of the best mode, and are not intended to limit the applicability or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Various representative implementations of the present invention may be applied to any system for providing botanically-sourced (or botanically-derived) topical emollient sanitizing compositions. As used herein, the terms “derivative,” “extract,” “source,” or any combinatorial, variational or contextual equivalent thereof, are generally intended to include anything that may be regarded as at least being susceptible to characterization as, or generally referring to, one or more compounds as they exist in nature and/or chemically altered forms thereof.

As used herein, the terms “sanitize”, “sanitizing”, “sanitization”, or any combinatorial, variational or contextual equivalent thereof, are generally intended to include anything that may be regarded as at least being susceptible to characterization as, or generally referring to, a material having anti-microbial, bactericidal, antiviral and/or disinfectant activity, including the prevention and/or inhibition of growth and/or killing of bacteria, viruses, fungi of any kind and by any mechanism of action or system of activation.

As used herein, the terms “topical formulation”, “topical composition”, or any combinatorial, variational or contextual equivalent thereof, are generally intended to include anything that may be regarded as at least being susceptible to characterization as, or generally referring to, a cosmetic, a pharmaceutical, a topical medicament, a personal care product, a shampoo, a conditioner, a leave-in conditioner, a hair product, a hair-styling product, a mousse, a nail product, a skin product, a moisturizer, a soap, a body wash, a shaving product, a gel, a lotion, a cream, an ointment, a fragrance, a foundation, a mascara, a gloss, a lip balm, a lip stick, a lip liner, an eye liner, a cosmetic remover, a cleanser, a scrub, a wax, a spray, a foam, a paste, a solid, a liquid, a towelette, a napkin, a feminine hygiene product, a facial mask, a sanitizer, a balm, a detergent, an ultraviolet radiation absorber, a sunscreen, a suntan lotion, a sun block, a sun tan oil, a repellant, a skin astringent, a skin toner, a skin freshener, and/or the like.

As used herein, the term “topical application” or any combinatorial, variational or contextual equivalent thereof, is generally intended to include anything that may be regarded as at least susceptible to characterization as, or generally referring to, use of a topical composition or topical formulation on or in conjunction with the hair, skin or a component layer of the skin, nails and/or any surface of any subject (animate or otherwise) or object.

As used herein, the terms “subject”, “user”, or any combinatorial, variational or contextual equivalent thereof, are generally intended to include anything that may be regarded as at least being susceptible to characterization as, or generally referring to, an animal, a human, and/or any at least partially porous surface (living or inanimate) suitably adapted for receiving a topical application of a topical formulation or topical composition.

As used herein, the term “botanical”, including any combinatorial, variational or contextual equivalent thereof, generally refers to anything that may be regarded as at least being susceptible to characterization as, or generally indicative of, a material or combination of materials that may be sourced, liberated or derived (chemically or otherwise) from a naturally occurring resource. While the use of the term “botanical” (and equivalents thereof) may certainly be intended to reference the vernacular meaning ordinarily ascribed to the term as designating properties of or relating to plant life, the scope of the term “botanical” (as used herein) should be understood to extend to various other “naturally occurring” materials that may be sourced or otherwise liberated from any material that at one time comprised living matter (plant-based or otherwise; e.g., petrolatum, mineral oil, etc.) and/or other mineral resources.

As used herein, the terms “organic”, “organic certification”, “organically derived” or any combinatorial, variational or contextual equivalent thereof, are generally intended to include anything that may be regarded as at least being susceptible to characterization as, or generally referring to, materials that have satisfied the criteria of a certification process generally imposed on producers of organic agricultural products. In general, any business supplying natural- and/or naturally-derived products may be certified, including seed suppliers, farmers, food processors, retailers and restaurants. Requirements vary from country to country, and generally involve production standards for growing, storage, processing, packaging and shipping that include, for example: (i) avoidance of most synthetic chemical inputs (e.g., fertilizer, pesticides, antibiotics, food additives, etc.), genetically modified organisms, irradiation, and the use of sewage sludge; use of farmland that has been free from chemicals for a number of years (e.g., often, three (3) or more); keeping detailed written production and sales records (e.g., audit trail); maintaining strict physical separation of organic products from non-certified products; submitting to periodic on-site inspections; and other procedures/requirements prescribed by various organic certifying authorities.

As used herein, the terms “improvement”, “improved”, “benefit”, “beneficial”, or any combinatorial, variational or contextual equivalent thereof, may mean an increased incidence in observance of a favorable property or a decreased incidence in observance of an unfavorable property. That notwithstanding, these same terms may also refer to a decrease in incidence in observance of what may correspond in alternative, conjunctive or sequential applications to an otherwise favorable property or the increase in incidence in observance of an otherwise unfavorable property.

A detailed description of a representative embodiment, namely a composition and method for providing a botanically-sourced or botanically-derived topical emollient sanitizing composition, is provided as a specific enabling disclosure that may be generalized to any application of the disclosed compositions and methods in accordance with various representative aspects of the present invention.

The present invention relates to botanically-sourced and botanically-derived topical emollient compositions having disinfectant properties. In a representative embodiment of the present invention, a composition may comprise a sanitizing component and a botanically-sourced or botanically-derived emollient component.

In accordance with various aspects of the present invention, a suitable emollient sanitizing composition may comprise an anti-microbial sanitizer and a botanically-sourced or botanically-derived emollient. The emollient sanitizing composition may representatively be applied to a topical surface of a subject, such as the skin of a mammalian subject. The emollient sanitizing composition may then be rubbed on the skin until the emollient components are substantially absorbed and/or the sanitizing components are substantially evaporated or otherwise dissipated. This process may then be repeated with the subject as frequently as indicated to provide both sanitizing function to the applied surface as well as improved moisturizing function not found with conventional hand sanitizing formulations. Representative benefits may include, for example, improved moisture retention, soft-feel, increased substantivity, and/or the like. Additionally, in various aspects in accordance with representative embodiments of the present invention, the disclosed emollient sanitizing compositions may be implemented to at least maintain or otherwise improve lipid profiles of the skin of a mammalian subject while concurrently, conjunctively or sequentially sanitizing the skin's surface after application.

In accordance with various aspects of the present invention, an anti-microbial sanitizer may comprise any composition suitably adapted to provide an at least partially disinfecting function when topically applied to a surface. In a representative embodiment of the present invention, a suitable anti-microbial sanitizer may at least partially penetrate cell walls of bacteria and denature proteins within the cells. This denaturing generally operates to interrupt the life-cycle of the bacterium, thereby killing it.

In accordance with various aspects of the present invention, representative sanitizing compositions may include alcohols and/or other disinfectant/anti-microbial formulations and/or botanical extracts (or derivatives thereof) including, but not limited to, chlorhexidine gluconate, benzalkonium chloride, iodine, grapeseed oil, lemon juice, tea tree oil, citronellol, camphor oil, cade oil, eucalyptol, clove oil, and/or the like. In a representative embodiment of the present invention, an anti-microbial sanitizer may comprise a lower hydrocarbon chain alcohol, such as a C₁₋₄ alcohol. In another representative embodiment of the present invention, the alcohol may comprise ethanol, 2-propanol, and/or n-propanol. In yet another representative embodiment of the present invention, an anti-microbial sanitizer may further comprise a dermatological active agent, a pharmaceutical composition, an antibiotic, a bactericidal agent, an antiseptic agent, a disinfectant agent, an antiviral agent, a nitrogenous cationic surface-active agent, a fruit juice, a fruit extract, and/or the like.

It should be appreciated that in representative embodiments of the present invention, a suitable anti-microbial sanitizer may comprise a combination of water and alcohol, such as an ethanol azeotrope. In yet a further representative embodiment of the present invention, ethanol may be present in concentrations between about 60%-95% (wt/wt).

With respect to various representative aspects of the present invention, an anti-microbial sanitizer may be suitably adapted for combination with a botanically-sourced or botanically-derived emollient composition to provide both sanitizing and moisturizing function.

A representative emollient composition in accordance with various aspects of the present invention may comprise any components that are suitably adapted for providing moisture retention, reduction of transepidermal water loss (TEWL), smooth feel, softness, increased substantivity, and/or the like. Additionally, representative emollient compositions may be employed to soften or smooth the skin by reducing roughness, cracking, irritation, and/or the like. In representative and exemplary aspects, a botanically-source or botanically-derived emollient may be selected to provide a lipid profile substantially similar to that of mammalian sebum (e.g., human sebum).

Additionally, in accordance with various aspects of the present invention, botanical emollient compositions may include bland, fatty, oleaginous substances that smooth the skin by penetration into the surface layers of skin tissue through the action of rubbing and massaging after application by the user.

Sources of representative botanical emollients in accordance with various aspects of the present invention include a number of fatty acids, wax esters, sterols, and/or the like (e.g., jojoba oil, shea oil, macadamia oil, rice bran wax, African dry zone mahogany seed oil, custard apple seed oil, sugar apple seed oil, common seabuckthom seed oil, and/or the like—including derivatives thereof). Fatty acids generally comprise aliphatic hydrocarbons or other organic chains with carboxylic substitutes therein, typically having between 8 and 24 carbon atoms in the backbone. Fatty acids generally include at least one of stearic acid, oleic acid, myristic acid and palmitic acid. Other typical fatty acids include linoleic acid, behenic acid, arachidic, lignoceric, and other common fatty acids of the general formulae CₙH₍₂ₙ₊₁₎COOH, CₙH₍₂ₙ₋₁₎COOH or CₙH₍₂ₙ₋₃₎COOH where “n” is an integer from 8 to 24.

Fatty alcohols have been found to be less sticky and less heavy than many other fatty materials (such as fatty acids), and are frequently used to improve the viscosity and stability of lotions and creams. Representative examples of fatty alcohols which find use in cosmetics and personal care products are cetyl alcohol, lauryl alcohol, stearyl alcohol, and oleyl alcohol.

Additional examples of representative emollients include fatty esters. One of the qualities of fatty esters is that they generally do not feel as oily to the touch as some other types of fatty emollient ingredients. Representative examples include isopropyl palmitate, isopropyl myristate, myristyl propionate, ethylhexyl palmitate, and glyceryl stearate.

In a representative embodiment of the present invention, a topical emollient composition may be derived or extracted from a botanical source. In another representative embodiment of the present invention, a botanically-sourced (or botanically-derived) emollient composition may include fatty acids, esters of fatty acids, alkoxylated fatty acids, fatty alcohols, esters of fatty alcohols, esters of fatty alcohols with fatty acids, sugar alcohols, isopropyl esters, wax esters and/or combinations thereof derived from the seed oil of the jojoba plant (Simmondsia chinensis), such as, for example: raw and/or refined jojoba oil, a jojoba ester, hydrogenated jojoba oil, a jojoba hydrolysate, a hydrolyzed jojoba ester, a jojoba alcohol, an alkoxylated jojoba wax, an alkoxylated and at least partially hydrogenated jojoba wax, an alkoxylated product of jojoba oil interesterified with hydrogenated jojoba oil, an isopropyl jojobate, and/or the like. In a representative embodiment of the present invention, a botanical emollient composition may include jojoba oil and/or derivatives including hydrogenated jojoba oil, isopropyl jojobate, jojoba alcohol, jojoba esters, and/or hydrolyzed jojoba esters.

Jojoba oil and jojoba derivatives according to the present invention may comprise about more than 6% unsaponifiables. The term “unsaponifiable” generally refers to a portion of the fat and/or oil (or in the case of jojoba, a wax ester) that is not susceptible to saponification. Generally, unsaponifiable materials typically comprise components that are naturally found in the fats and/or oils, such as phenols, tocopherols, triterpenes, steroids, sterols, hydrocarbons such as squalene, alcohols, and/or the like. Unsaponifiable material may be retained with the saponified material through an in situ saponification process, in which the unsaponifiable material is generally not removed and/or separated from the saponified material.

A saponification reaction may be accomplished by the hydrolysis of an ester under basic conditions, such as in the presence aqueous alkali metal hydroxides (e.g., NaOH, LiOH, KOH, CaOH, MgOH, and/or the like) to form an alcohol and a salt of a carboxylic acid. In a representative embodiment of the present invention, in situ saponification may be affected through a base-catalyzed hydrolysis reaction between jojoba oil (a liquid wax ester at room temperature) and/or jojoba derivatives and an alkyl alcohol.

The products of the in situ saponification of jojoba oil typically comprise jojoba hydrolysates, which include a mixture of: (i) salts of jojoba fatty acids (saponifiables); and (ii) non-polar, lipophilic materials (unsaponifiables), with the possibility of other materials also present, depending on the source, state and form of the initial reactant (include residual jojoba wax ester).

The in situ production of unsaponifiable materials in tandem with saponified material from fats, oils and/or their derivatives having high levels of unsaponifiables in accordance with various aspects of the present invention may provide various benefits in compositions prepared for topical application to the skin of a subject. These benefits may include, for example, moisturization, a desirable texture, substantivity, resistance to wear, and water- and/or rinse-resistance. The presence of high unsaponifiables may also provide occlusive properties to the topical formulation where water is maintained in the skin, providing retained softness and smoothness.

In a representative embodiment of the present invention, botanical emollient material comprising in situ products of saponification may function to preserve superior skin feel and substantivity generally attributed to the polar hydrophilic properties of, for example, jojoba oil components.

Additionally, emollient materials in accordance with various representative embodiments of the present invention may generally form stable emulsions more readily than those incorporating naturally occurring jojoba oil. In another representative embodiment of the present invention, representative emollient materials may also impart an improved lipid profile to the skin of a subject after multiple treatments, as compared with conventional skin sanitizers.

It should further be appreciated that in accordance with various aspects of the present invention, botanical emollient components of the disclosed compositions may be employed to at least partially reconstitute the lipid profile of the stratum corneum barrier of the skin by providing lipids and derivatives thereof that chemically resemble human sebum. Additionally, in a representative embodiment of the present invention, botanical emollient compositions in accordance with the present invention generally provide superior smoothness and substantive skin-feel by being absorbing into the skin and/or maintaining a persisting presence on the surface of the skin.

Representative botanical emollient and anti-microbial sanitizer compositions may be formulated in any suitable manner. For example, a suitably adapted anti-microbial sanitizer may comprise a substantially transparent, translucent and/or opaque liquid. Additionally, a suitably adapted botanical (‘sourced’ or ‘derived’) emollient component of the composition may comprise carrier particles, such as natural and/or synthetic emollient beads. Representative carrier particles may comprise any suitable synthetic and/or natural components. For example, carrier particles at least partially comprising natural emollient beads may be produced from combinations of fatty alcohols, isopropyl esters, wax esters, and/or the like, obtained from jojoba oil and/or jojoba derivatives. Carrier particles comprising at least partially synthetic beads may also include components such as polyethylene, petrolatum, ethylhexyl palmitate, and/or the like.

Additionally, it should be appreciated that representative carrier particles may include any suitable texture, size, shape, and/or the like. For example, suitably adapted carrier particles may comprise visible mono-sized beads having a diameter on the order of at least about 50 microns to more than about 5,000 microns. In a representative embodiment of the present invention, suitably configured carrier particles may comprise beads that are generally soft and adapted to rub into the skin while leaving substantially no debris behind. In another representative embodiment, suitably configured carrier particles may be adapted to carry active ingredients. In yet a further representative embodiment of the present invention, carrier particle beads may be comprised of materials that are solid at room temperature and configured in various shapes and/or sizes.

Additionally, carrier particle beads may provide color and/or texture so as to be visible in product suspension. In another representative embodiment of the present invention, the color and/or texture of carrier particles may at least partially assist the user in topical application or delivery of a botanical component of an emollient sanitizing composition to a surface via visual verification of deposition.

Botanical emollient sanitizing compositions for topical use, in accordance with various representative aspects of the present invention, may be formulated in any suitable manner. For example, in a representative embodiment, an anti-microbial sanitizer and a botanical emollient may be combined with one or more additives. Representative additives, in accordance with various aspects of the present invention, may include, for example: a coloring agent, a dye, a color shifting pigment, a preservative, a pH adjusting material, a pH buffering agent, a thickening agent/polymer, a fragrance material, a polar extract of a fragrance material, water, a polyacrylic acid, polymer, a sugar alcohol, glitter, a special effect pigment, a vitamin, a provitamin, an amino acid, a protein, a peptide, a peptide complex, an active agent, and/or the like.

In another representative embodiment of the present invention, a botanical emollient sanitizing composition may be formulated with glycerine (alternatively spelled “glycerin”, but equivalent to glycerine in material respect). As a humectant, glycerine generally functions to enhance or at least substantially maintain substantivity of an emollient composition. A humectant is generally regarded as a hygroscopic substance and is often a molecule with several hydrophilic groups, most often hydroxyl groups; however, amines and carboxyl groups (sometimes esterified) may be employed as well. Humectants typically demonstrate an affinity to form hydrogen bonds with molecules of water. Humectants are often found in many cosmetic products where moisturization is desired, including, for example, moisturizing treatments for the hair. Representative examples of humectants include glycerine, propylene glycol (E 1520), butylene glycol, polyglycerol, polyglycerol esters, and glyceryl triacetate (E1518) and the like. Others may include polyols like sorbitol (E420), xylitol and maltitol (E965), or polymeric polyols like polydextrose (E1200) or natural extracts like quillaia (E999), or lactic acid or urea, and the like.

The property of a material demonstrating “substantivity” may generally be regarded as its propensity to persist and reside on a surface to which it is applied. With enhanced substantivity, the combination of glycerine with an emollient may provide improved moisture retention properties.

In accordance with various representative aspects of the present invention, a botanical emollient sanitizing composition may be formulated into one or more commercial formulations, as generally illustrated by the Examples given below. The percentages detailed below should be regarded as approximate.

A representative antibacterial emollient sanitizing composition may be formulated as a hand sanitizer gel in accordance with the following:

### Example 1

| Phase | Common Designation | INCI Designation | % (wt/wt) |
|---|---|---|---|
| A | Deionized Water | Water | 6.70 |
| | FLORASOLVS | Jojoba Oil PEG-150 Esters | 0.10 |
| | (International Flora | | |
| | Technologies, Ltd. | | |
| | {“Floratech”}, Chandler, AZ, | | |
| | USA) PEG-150 | | |
| | Hydrogenated Jojoba | | |
| | CARBOPOL (Lubrizol | Acrylates/C10-30 Alkyl Acrylate | 25.00 |
| | Advanced Materials, Inc., | Crosspolymer; | |
| | Cleveland, Ohio, USA) ETD | Water; | |
| | 2020 (1.0% in water); | Methylchloroisothiazolinone and | |
| | KATHON (Rohm & Haas | Methylisothiazolinone; and | |
| | Company, Philadelphia, PA, | Triethanolamine | |
| | USA) CG at 0.01%; and | | |
| | Triethanolamine (TEA) | | |
| | titrated to pH = 6.5 | | |
| | Glycerine | Glycerin | 0.70 |
| | SIMULGEL (Societe | Acrylamide/Sodium | 2.00 |
| | D'Exploitation de Produits | Acryloyldimethyltaurate Copolymer | |
| | Pour Les Industries | (and) Isohexadecane (and) | |
| | Chimiques Seppic, Paris, | Polysorbate 80 | |
| | France) 600 | | |
| B | Ethanol SDA 40-2 (200 | Alcohol | 61.00 |
| | proof) | | |
| | FLORAESTERS (Floratech, | Jojoba Esters (and) Isopropyl Jojobate | 2.00 |
| | Chandler, AZ, USA) IPJ | (and) Jojoba Alcohol | |
| | FLORAMAC (Floratech | Ethyl Macadamiate | 1.40 |
| | Chandler, AZ, USA) 10 | | |
| | Bisabolol | Bisabolol | 0.10 |
| | FLORAESTERS K-100 | Hydrolyzed Jojoba Esters (and) | 0.10 |
| | | Jojoba Esters (and) Water | |
| | Dermolene (Aston | Olea Europaea (Olive Oil) | 0.10 |
| | Chemicals, Ltd., Aylesbury, | Unsaponifiables | |
| | UK) | | |
| C | FLORASOMES (Floratech, | Jojoba Esters (and) Tocopheryl | 0.80 |
| | Chandler, AZ, USA) Jojoba | Acetate | |
| | SMS White (natural) 10% | | |
| | Vitamin E | | |
| | | TOTAL: | 100% |

Formulation Procedure:

1. In a suitable vessel, add water of Phase A. Add CARBOPOL ETD/KATHON/TEA gel to the water and mix until uniformly dispersed. Add FLORASOLVS PEG-150 Hydrogenated Jojoba and mix with propeller agitation until clear.

2. Add glycerin to main mixing vessel and stir until uniform. Add SIMULGEL 600 to the main mixing vessel and stir until a uniform texture results.

Referring now to Example 2, in another representative embodiment of the present invention, FLORAMAC 10 and Dermolene are excluded while additional FLORAESTERS K-100 may be used to modify pH. Fragrance and preservative may also be added.

### Example 2

| | | % |
|---|---|---|
| Common Designation | INCI Designation | (wt/wt) |
| Deionized Water | Water | 8.65 |
| FLORASOLVS PEG-150 | Jojoba Oil PEG-150 Esters | 0.10 |
| Hydrogenated Jojoba | | |
| CARBOPOL ETD 2020 | Acrylates/C10-30 Alkyl | 20.00 |
| (1.0% in water); | Acrylate Crosspolymer; | |
| KATHON CG at 0.01%; | Water; | |
| and Triethanol- | Methylchloroisothiazolinone | |
| amine titrated to | and Methylisothiazolinone; | |
| pH = 6.5 | and Triethanolamine | |
| Glycerine | Glycerin | 5.00 |
| SIMULGEL 600 | Acrylamide/Sodium | 2.50 |
| | Acryloyldimethyltaurate | |
| | Copolymer (and) Isohexadecane | |
| | (and) Polysorbate 80 | |
| Ethanol SDA 40-2 | Alcohol | 61.00 |
| (200 proof) | | |
| FLORAESTERS IPJ | Jojoba Esters (and) Isopropyl | 1.50 |
| | Jojobate (and) Jojoba Alcohol | |
| Bisabolol | Bisabolol | 0.10 |
| FLORAESTERS K-100 | Hydrolyzed Jojoba Esters (and) | 0.10 |
| | Jojoba Esters (and) Water | |
| FLORASOMES Jojoba | Jojoba Esters (and) Tocopheryl | 0.50 |
| SMS 10% Vitamin E | Acetate | |
| FLORASOMES Jojoba | Jojoba Esters (and) Tocopheryl | 0.50 |
| MDS 10% Vitamin E | Acetate | |
| Fragrance | fragrance | 0.05 |
| | TOTAL: | 100% |

Another representative embodiment of the present invention may be illustrated in Example 3. In this representative formulation, the amount of CARBOPOL, glycerine, FLORAESTERS IPJ, SIMULGEL 600, FLORASOMES, TEA and preservative is reduced as compared to Example 2, while water is added to compensate for the decreased formulation volume. Optionally, fragrance may be excluded as well.

### Example 3

| | | % |
|---|---|---|
| Common Designation | INCI Designation | (wt/wt) |
| Deionized Water | Water | 18.70 |
| FLORASOLVS PEG-150 | Jojoba Oil PEG-150 Esters | 0.10 |
| Hydrogenated Jojoba | | |
| CARBOPOL ETD 2020 | Acrylates/C10-30 Alkyl | 15.00 |
| (1.0% in water); | Acrylate Crosspolymer; | |
| KATHON CG at 0.01%; | Water; | |
| and Triethanol- | Methylchloroisothiazolinone | |
| amine titrated to | and Methylisothiazolinone; | |
| pH = 6.5 | and Triethanolamine | |
| Glycerine | Glycerin | 1.40 |
| SIMULGEL 600 | Acrylamide/Sodium | 2.00 |
| | Acryloyldimethyltaurate | |
| | Copolymer (and) Isohexadecane | |
| | (and) Polysorbate 80 | |
| Ethanol SDA 40-2 | Alcohol | 61.00 |
| (200 proof) | | |
| FLORAESTERS IPJ | Jojoba Esters (and) Isopropyl | 1.00 |
| | Jojobate (and) Jojoba Alcohol | |
| Bisabolol | Bisabolol | 0.10 |
| FLORAESTERS K-100 | Hydrolyzed Jojoba Esters (and) | 0.10 |
| | Jojoba Esters (and) Water | |
| FLORASOMES Jojoba | Jojoba Esters (and) Tocopheryl | 0.30 |
| SMS 10% Vitamin E | Acetate | |
| FLORASOMES Jojoba | Jojoba Esters (and) Tocopheryl | 0.30 |
| MDS 10% Vitamin E | Acetate | |
| | TOTAL: | 100% |

The formulation of Example 3 illustrates a representative embodiment that may serve to reduce stickiness and/or “tack” otherwise associated with conventional hand sanitizers. The representative formulation of Example 3 may also reduce production costs. In the Examples disclosed supra, representative botanical emollients may comprise FLORAESTERS IPJ, FLORAMAC 10, FLORAESTERS K100, and FLORASOMES. FLORAESTERS IPJ are generally obtained from the product of incomplete saponification of jojoba oil (Simmondsia chinensis) yielding in approximately equal amounts: wax esters, jojoba alcohols, and isopropyl esters of jojoba fatty acids. FLORAMAC 10 corresponds to ethyl esters of macadamia oil (Macadamia integrifolia) fatty acids. Macadamia oil and FLORAMAC 10 are high in palmitoleic acid (C16:1)—a fatty acid know to be present as a significant fraction of human sebum. FLORAESTERS K100 corresponds to saponification products of jojoba oil in conjunction with unsaponifiable material produced from that reaction. Specifically, FLORAESTERS K100 is generally comprised of potassium salts of jojoba fatty acids, the corresponding jojoba free fatty alcohols, and a small amount of residual jojoba wax ester. FLORASOMES generally comprise jojoba oil randomized with fully hydrogenated jojoba oil, yielding wax esters of varying degrees of unsaturation.

Unsaturated alcohols of human sebum have not been fully characterized previously, however, somewhat similar alcohols have been observed in the seed oil of Simmondsia chinsensis. FLORAESTERS K100 provides a significant source of unsaturated alcohols derived from botanically-sourced jojoba oil. Human sebum also contains wax esters, with more active sebaceous glands producing sebum lipids with a higher proportion of C16:1 straight chain fatty acids. Similar wax esters may be obtained from, for example, FLORAESTERS IPJ and FLORASOMES—both representing derived compounds from botanically-sourced jojoba oil. Additionally, a C16:1 lipid profile similar to that of mammalian sebum may be obtained with FLORAMAC 10—a derived material of botanically-sourced macadamia oil.

Disclosed botanical topical emollient sanitizing compositions, in accordance with various representative embodiments of the present invention, may be formulated for delivery via in any suitable manner, such as with a towelette, a pre-saturated towelette, a wipe, a napkin, a feminine hygiene product, a spray, a liquid, a gel, a cream, a lotion, a foam, a paste, a facial mask, a soap, and/or any other suitable formulation vehicle.

In accordance with various aspects of the present invention, representative topical emollient sanitizing compositions may be formulated in a towelette, where the towelette may be suitably adapted to absorb and/or retain the emollient sanitizing composition. Additionally, a towelette may be implemented so as to prevent drying or evaporation of an emollient sanitizing composition. The material of the towelette may also be disposable, washable, reusable, and/or the like.

In a further representative embodiment of the present invention, a topical emollient sanitizing composition may be added to the material of a towelette in sufficient quantity to dampen the towelette material so that the composition may be transferred to the skin or other surface of application upon contact with the towelette. The user may rub and/or wipe the towelette on the skin until emollient sanitizing composition is substantially absorbed. Debris on the surface of the skin may be further removed by contact of the towelette material on the skin.

In another representative embodiment of the present invention, an emollient sanitizing composition may be formulated to produce a sanitizing and moisturizing detergent for use as, for example, an anti-microbial soap for the removal of apolar bacteria, dirt, grease, oils, and/or the like from skin. Apolar materials may be lifted from the skin by association with micelles formed with soap molecules for subsequent washing away with water.

The presence of representatively disclose emollient sanitizing formulations in conjunction with soap generally provides a soft substantive skin-feel and reduces dermatitic symptoms associated with frequent hand washing. In another representative embodiment of the present invention, an emollient sanitizing soap may comprise the products of the saponification of a variety of botanical and/or synthetic fats. In a further representative embodiment, an emollient sanitizing soap may be provided as a solid, liquid, foam, spray, gel, cream, lotion, and/or the like.

Representative botanical emollient sanitizing compositions in accordance with the present invention may also be formulated with a skin toner. Skin toners generally function to sanitize the skin and diminish the size of pores. Conventional skin toners may vary according to their concentration of alcohol. For example, an astringent is a type of skin toner that generally comprises alcohol up to about 60%. A Skin tonic, on the other hand, is a type of skin toner that generally comprises less alcohol than astringents and may have up to about 20% alcohol. Additionally, a skin refresher is a type of skin toner that generally comprises the least amount of alcohol—on the order of about less than 10% alcohol.

In accordance with various representative aspects of the present invention, botanical emollient sanitizing compositions may increase the range of applications for skin sanitizing compositions that may be suitably formulated for use by persons in which conventional sanitizing formulations may be contra-indicated. For example, individuals with sensitive skin, eczema, shingles, the skin of infants or young children, and/or the like, may experience significant adverse dermatitic symptoms upon repeated use of conventional sanitizer products. Individuals with these same dermatological conditions generally do not experience such symptoms, or at least observe a reduction of symptoms, after use of botanical emollient sanitizing compositions in accordance with representative embodiments of the present invention. Furthermore, botanical emollient sanitizing compositions in accordance with the present invention may also be suitably adapted for use on artificial (e.g., inanimate) surfaces that require sanitization without drying effects.

In a representative embodiment of the present invention, an emollient sanitizing composition may be employed to alleviate dermatitis, such as acquired occupational hand dermatitis, and/or the like. In a recent study of individuals having a history of chronic hand dermatitis for an average of thirteen (13) years of duration prior to the study due to repeated daily use (>10 times per day) of conventional commercial alcohol gel sanitizing products, all fourteen (14) participants in the study exhibited substantial reduction of dermatitic symptoms after topical application of the emollient sanitizing composition corresponding to Example 1.

The study participants used the emollient sanitizing composition a minimum of eight (8) times per day over a fourteen (14) day period. For three (3) consecutive days prior to the study, the subjects cleansed their hands with CETAPHIL (Galderma Laboratories, L. P., Cham Switzerland) soap, which was used to replace their daily hand soap to provide a baseline reference. The use of other hand moisturizers and topical products (over-the-counter or prescription) were not permitted during the duration of the study. At the beginning of the study, a physician's assessment was conducted to evaluate abnormal skin symptoms. Additionally, bio-instrumental evaluation for evaporative skin moisture loss (TEWL) was performed on the dorsal side of the subjects' hands (commonly referred to as the “back” or “topside” of the hand), as well as on the palmar side of the subjects' hands (commonly referred to as the “palm” or the “inner surface” of the hand). Subjects thereafter started a course of application of the emollient sanitizer composition corresponding to Example 1 a minimum of eight (8) times per day for a duration of fourteen (14) days.

TABLE 1

| | Baseline | | 14 Days Post-Treatment | | | |
|---|---|---|---|---|---|---|
| | x̅ | x̅ | x̅ | | x̅ | |
| | Dorsal | Palmer | Dorsal | | Palmer | |
| Erythema | 2.0 | 2.0 | <1.0 | | <1.0 | |
| Scaling | 2.0 | 2.0 | <1.0 | | <1.0 | |
| Fissuring | 1.0 | 1.0 | 0 | (healed) | 0 | (healed) |
| Xerosis | 2.0 | 2.0 | <1.0 | | <1.0 | |
| Edema | 0 (absent) | 0 (absent) | 0 | (no change) | 0 | (no change) |
| Vesicula- | 0 (absent) | 0 (absent) | 0 | (no change) | 0 | (no change) |
| tion | | | | | | |
| Lichenifi- | 0 (absent) | 0 (absent) | 0 | (no change) | 0 | (no change) |
| cation | | | | | | |

Table 1 representatively illustrates a physician's clinical assessments of the hands of study participants. The physician evaluated both dorsal and palmar sides of the hands before treatment and after fourteen (14) days of treatment. Subjects were evaluated for symptoms corresponding to various dermatological abnormalities, including, for example: erythema (e.g., redness of the skin caused by capillary congestion); scaling (e.g., flaking of the skin); fissuring (e.g., cracks in the skin that may bleed); xerosis (e.g., dry skin); edema (e.g., swelling of the skin); vesiculation (e.g., formation of blisters); and lichenification (e.g., the formation of thick, leathery skin, usually the result of constant scratching and rubbing). The physician used a scoring system corresponding to the following: 0=an absence of the condition; 1=mild incidence; 2=moderate incidence; 3=moderately severe incidence; and 4=very severe incidence. At day fourteen (14), all fourteen (14) subjects demonstrated clinical improvement (or at least no change with respect to edema, vesiculation and lichenification). Erythema, scaling, and xerosis went from a moderate score of 2 at baseline, to slight improvement following fourteen (14) days of product use. Notably, following two (2) weeks of product use, fissures that subjects presented with at baseline were healed!

Over a fourteen (14) day period, the hands of study participants were clinically assessed for TEWL, an objective measurement of moisture loss from the skin due to, for example, evaporation. TEWL values are related to the degree of perturbation of the stratum corneum with a decrease in TEWL values denoting improved function of this barrier layer of the skin. These measurements were performed using a TM 300 Tewameter (Courage-Khazaka, Koln, Germany).

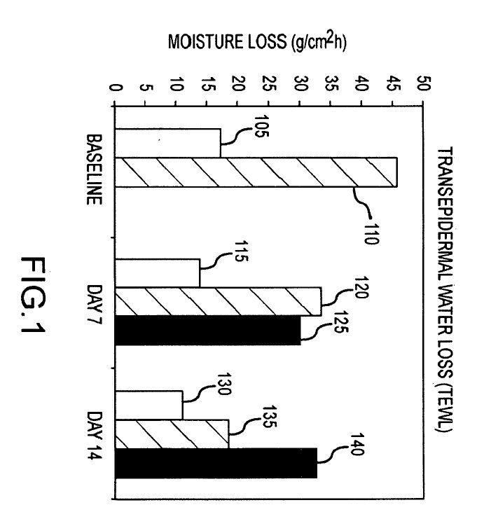

Referring to FIG. 1, differences in TEWL with the use of an emollient sanitizing composition (Example 1) measured on dorsal 115, 130 and palmar 120, 135 surfaces of hands of study participants as compared to baseline 105, 110 (dorsal and palmar, respectively) over time were observed. TEWL was measured in units of grams of water lost per square-centimeter per hour (g/cm² h). At baseline (prior to application of the emollient sanitizer composition corresponding to Example 1), the mean dorsal TEWL 105 was 17.12 g/cm² h and the mean palmar TEWL 110 was 45.71 g/cm² h. These values decreased to 13.8 g/cm² h and 33.5 g/cm² h after seven (7) days of regular application of emollient sanitizer (dorsal 115 and palmar 120, respectively). This corresponded to a 19.39% reduction of dorsal TEWL and a 26.71% reduction of palmar TEWL from baseline thru day seven (7).

After two (2) weeks of application, dorsal 130 and palmar 135 values decreased again to 11.04 g/cm² h and 18.51 g/cm² h, respectively. This corresponded to a 20.00% reduction of dorsal TEWL and a 44.75% reduction of palmar TEWL from day seven (7) thru day fourteen (14). The aggregate effect observed over the course of the entire study corresponded to a 35.51% reduction of dorsal TEWL and a 59.51% reduction of palmar TEWL from baseline thru day fourteen (14).

To account for environmental factors that could potentially result in errant increases or decreases in TEWL, as well as to verify that the Tewameter probe was functioning properly, untreated sites 125, 140 were measured on medial inner-wrist patches for each subject on day seven (7) and day fourteen (14), respectively. The mean value for TEWL control readings on wrist patches at seven (7) days 125 corresponded to 30.00 g/cm² h. The mean value for TEWL control readings on wrist patches at fourteen (14) days 140 was 32.5 g/cm² h. Notably, even with slight increase in control measurements of TEWL over the duration of the study, both dorsal and palmar TEWL readings were dramatically reduced.

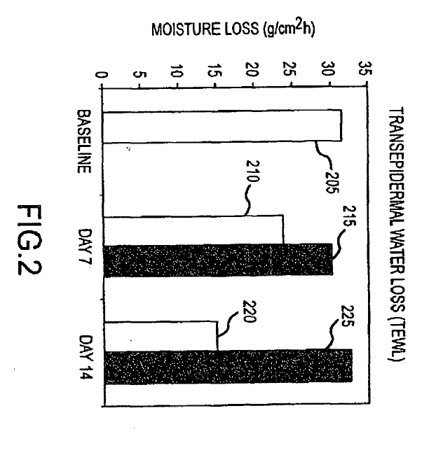

In terms of statistical significance, Student's t-distributions were calculated which demonstrated: a less than 2% probability that the reduction in mean dorsal TEWL values between baseline and seven (7) days occurred as a matter of random chance; a less than 0.1% probability that the reduction in mean palmar TEWL values between baseline and seven (7) days occurred as a matter of random chance; less than 1% probability that the reduction in mean dorsal TEWL values between baseline and fourteen (14) days occurred as a matter of random chance; and less than 0.1% probability that the reduction in mean palmar TEWL values between baseline and fourteen (14) days occurred as a matter of random chance. Referring to FIG. 2, differences in TEWL with the use of an emollient sanitizing composition (Example 1) were measured as averages of combined mean dorsal and mean palmar values 210, 220 compared to baseline 205 over time were observed. At baseline (prior to application of the emollient sanitizer composition corresponding to Example 1), the average combined mean dorsal and palmar TEWL 205 was 31.41 g/cm² h. This value decreased to 23.68 g/cm² h after seven (7) days of regular application of emollient sanitizer (dorsal and palmar combined 210). This corresponded to a 24.61% reduction of dorsal and palmar TEWL from baseline thru day seven (7). After two (2) weeks of application, the combined dorsal and palmar value 220 decreased again to 14.78 g/cm² h. This corresponded to a 37.58% reduction of combined dorsal and palmar TEWL from day seven (7) thru day fourteen (14). The aggregate effect observed over the course of the entire study corresponded to a 52.94% reduction of combined dorsal and palmar TEWL from baseline thru day fourteen (14).

Again, to account for environmental factors that could potentially result in errant increases or decreases in TEWL, as well as to verify that the Tewameter probe was functioning properly, untreated sites 215, 225 were measured on medial inner-wrist patches for each subject on day seven (7) and day fourteen (14), respectively. The mean value for TEWL control readings on wrist patches at seven (7) days 215 corresponded to 30.00 g/cm² h. The mean value for TEWL control readings on wrist patches at fourteen (14) days 225 was 32.5 g/cm² h. Notably, even with slight increase in control measurements of TEWL over the duration of the study, the combined averages of mean dorsal and palmar TEWL readings were dramatically reduced.

In terms of statistical significance, Student's t-distributions were calculated which demonstrated a less than 0.01% probability that the reduction in combined average of mean dorsal and palmar TEWL values between baseline and seven (7) days occurred as a matter of random chance, and a less than 0.01% probability that the reduction in combined average of mean dorsal and palmar TEWL values between baseline and fourteen (14) days occurred as a matter of random chance.

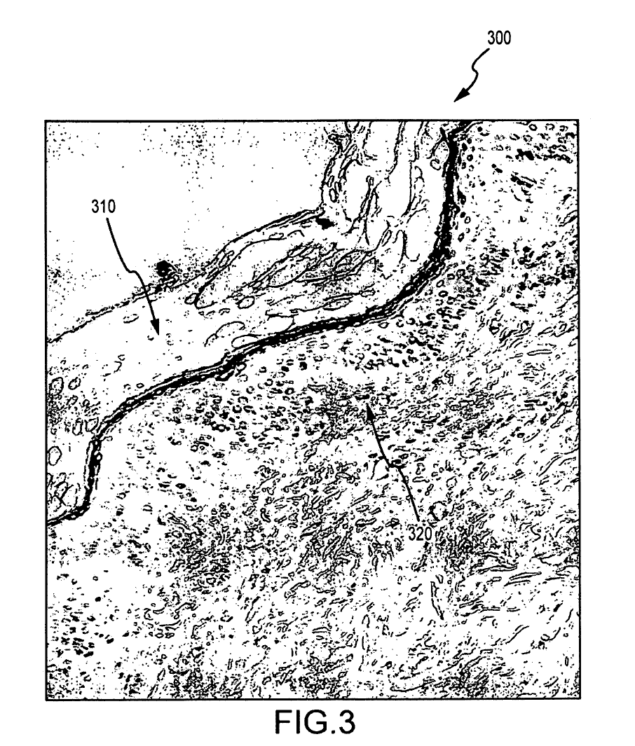

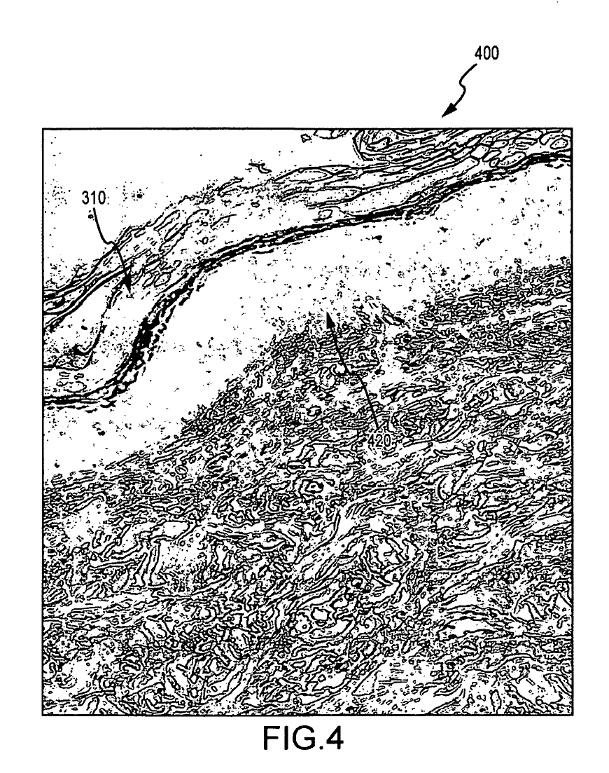

FIGS. 3 and 4 illustrate histopathology associated with a representative dermatological inflammatory process (i.e., contact dermatitis) both before and following application of an emollient sanitizer composition corresponding to Example 1. FIG. 3 corresponds to baseline photomicrograph results of a 3 mm punch biopsy of untreated skin stained with Hematoxylin and Eosin at a magnification of 400×. The histology depicts a thickening of the stratum corneum 310 and a mild-moderate inflammatory infiltrate of polymorphonuclear leukocytes (PMN's; i.e., white blood cells) 320 in the basal portion of the epidermis. This histological evaluation is consistent with common signs and symptoms associated with hand irritant contact dermatitis (e.g., redness, dryness, and fissuring).

FIG. 4 corresponds to measurement at fourteen (14) days post-treatment following use of the emollient sanitizing formulation of Example 1. Again, the photomicrograph was made from a 3 mm punch biopsy stained with Hematoxylin and Eosin at 400× magnification. (The vacuolization observed as open areas in the stratum corneum 310 depicted in FIGS. 3 and 4 correspond to artifacts of the method employed to prepare the cross-sectional samples for photomicrography and, as such, represent no difference between the photomicrographs.) The histology of FIG. 4 demonstrates a less thick stratum corneum 310 and less inflammatory (PMN) infiltrate 420 residing in the basal portion of the epidermis. These findings are consistent with resolution of inflammatory hand irritant contact dermatitis (e.g., reduction in redness, dryness, and fissuring).

Accordingly, emollient sanitizing compositions corresponding to various representative embodiments of the present invention generally provide a demonstrated reduction in transepidermal water loss, increase in substantivity and smooth skin-feel, as well as anti-inflammatory activity useful for the mitigation of adverse dermatitic symptoms.

In the foregoing specification, the invention has been described with reference to specific exemplary embodiments; however, it will be appreciated that various modifications and changes may be made without departing from the scope of the present invention as set forth herein. The specification is to be regarded in an illustrative manner, rather than a restrictive one and all such modifications are intended to be included within the scope of the present invention. Accordingly, the scope of the invention should be determined by the claims and their legal equivalents rather than by merely the examples described above.

For example, the steps recited in any method or process embodiment may be executed in any order and are not limited to the specific order presented in the claims. Additionally, the components and/or elements recited in any apparatus or composition embodiment may be assembled or otherwise operationally configured in a variety of permutations to produce substantially the same result as the present invention and are accordingly not limited to the specific configuration recited in claims.

Benefits, other advantages and solutions to problems have been described above with regard to particular embodiments; however, any benefit, advantage, or solution to a problem or any element that may cause any particular benefit, advantage, or solution to occur or to become more pronounced are not to be construed as critical, required, or essential features or components of the invention.

As used herein, the terms “comprising”, “having”, “including” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

We claim:

1. A composition for topical application to the skin of a mammalian subject, said composition comprising an anti-microbial sanitizer and at least one of a botanically-sourced and a botanically-derived emollient, wherein said emollient has a lipid profile substantially similar to that of mammalian sebum.

2. The composition of claim 1, wherein said anti-microbial sanitizer comprises at least one of an alcohol, chlorhexidine gluconate, benzalkonium chloride, iodine, grapeseed oil, lemon juice, tea tree oil, citronellol, camphor oil, cade oil, eucalyptol, clove oil, a dermatological active agent, a pharmaceutical composition, an antibiotic, a bactericidal agent, an antiseptic agent, a disinfectant agent, an antiviral agent, a nitrogenous cationic surface-active agent, a fruit juice, and a fruit extract.

3. The composition of claim 2, wherein said alcohol comprises at least one of ethanol, isopropyl alcohol, and a denatured alcohol.

4. The composition of claim 1, wherein said composition is certified as organic.

5. The composition of claim 1, wherein said anti-microbial sanitizer comprises at least one of: at least about 60% (wt/wt) ethanol, and at least about 60% (v/v) ethanol.

6. The composition of claim 1, wherein said emollient comprises at least one of jojoba oil, an extract of jojoba, a derivative of jojoba oil, a derivative of a jojoba extract, and a humectant.

7. The composition of claim 6, wherein the derivative of at least one of said jojoba oil and said jojoba extract comprise at least one of a jojoba ester, hydrogenated jojoba oil, a jojoba hydrolysate, a hydrolyzed jojoba ester, a jojoba alcohol, an alkoxylated jojoba wax, an alkoxylated and at least partially hydrogenated jojoba wax, an alkoxylated product of jojoba oil interesterified with hydrogenated jojoba oil, and an isopropyl jojobate.

8. The composition of claim 6, wherein said emollient further comprises at least one of: about 5%-35% (wt/wt) water; about 0.1% (wt/wt) PEG-150 hydrogenated jojoba; about 0.15%-0.25% (wt/wt) polyacrylic acid polymer; about 0.5%-5% (wt/wt) glycerin; about 1%-2% (wt/wt) of at least one of isopropyl jojobate, a jojoba alcohol, a jojoba ester, and tocopherol acetate; about 1.4% (wt/wt) ethyl macadamiate; about 0.1% (wt/wt) of an unsaponifiable fraction of olive oil hydrolysate; about 0.1% (wt/wt) bisabolol; about 0.1% (wt/wt) of at least one of a hydrolyzed jojoba ester, a jojoba ester, and water; about 2%-2.5% (wt/wt) of at least one of acrylamide, sodium acryloyldimethyl taurate copolymer, isohexadecane, and polysorbate 80; about 0.6%-1% (wt/wt) of at least one of a jojoba ester, a hydrogenated jojoba oil, and tocopherol acetate; up to about 0.06% (wt/wt) triethanolamine; a fragrance; and a preservative.

9. The composition of claim 1, wherein said emollient comprises at least one of a carrier particle and glycerin.

10. The composition of claim 1, further comprising an additive selected from the group consisting of: a coloring agent, a dye, a color-shifting pigment, a preservative, a pH modifier, a weak base, a pH buffering agent, a thickening agent, a polymer, a fragrance, a polar extract of a fragrance, water, a polyacrylic acid, a sugar alcohol, glitter, a special effect pigment, a vitamin, a pro-vitamin, an amino acid, a protein, a peptide, a peptide complex, and an active agent.

11. The composition of claim 1, wherein said composition is formulated into at least one of a soap, a body wash, a stringent, a toner, a freshener, a gel, a towelette, a napkin, a feminine hygiene product, and a wipe.

12. The composition of claim 1, wherein said composition provides at least one of: improvement of the function of the stratum corneum, reduction in transepidermal water loss, improved substantive feel, and improved moisturizing feel.

13. A moisturizing and sanitizing composition for topical application to the skin of a mammalian subject, said composition comprising an anti-microbial sanitizer and an emollient selected from the group consisting of jojoba oil, a jojoba ester, hydrogenated jojoba oil, a jojoba hydrolysate, a hydrolyzed jojoba ester, a jojoba alcohol, an alkoxylated jojoba wax, an alkoxylated and at least partially hydrogenated jojoba wax, an alkoxylated product of jojoba oil interesterified with hydrogenated jojoba oil, an isopropyl jojobate, and a humectant.

14. The composition of claim 13, wherein the emollient's lipid profile substantially corresponds to the lipid profile of mammalian sebum.

15. The composition of claim 13, wherein said anti-microbial sanitizer comprises at least one of an alcohol, chlorhexidine gluconate, benzalkonium chloride, iodine, grapeseed oil, lemon juice, tea tree oil, citronellol, camphor oil, cade oil, eucalyptol, clove oil, a dermatological active agent, a pharmaceutical composition, an antibiotic, a bactericidal agent, an antiseptic agent, a disinfectant agent, a nitrogenous cationic surface-active agent, a fruit juice, and a fruit extract.

16. The composition of claim 15, wherein said alcohol comprises at least one of ethanol, isopropyl alcohol, and a denatured alcohol.

17. The composition of claim 13, wherein said composition is certified as organic.

18. The composition of claim 13, wherein said anti-microbial sanitizer comprises at least one of: at least about 60% (wt/wt) ethanol, and at least about 60% (v/v) ethanol.

19. The composition of claim 13, wherein said emollient further comprises at least one of: about 5%-35% (wt/wt) water; about 0.1% (wt/wt) PEG-150 hydrogenated jojoba; about 0.15%-0.25% (wt/wt) polyacrylic acid polymer; about 0.5%-5% (wt/wt) glycerin; about 1%-2% (wt/wt) of at least one of isopropyl jojobate, a jojoba alcohol, a jojoba ester, and tocopherol acetate; about 1.4% (wt/wt) ethyl macadamiate; about 0.1% (wt/wt) of an unsaponifiable fraction of olive oil hydrolysate; about 0.1% (wt/wt) bisabolol; about 0.1% (wt/wt) of at least one of a hydrolyzed jojoba ester, a jojoba ester, and water; about 2%-2.5% (wt/wt) of at least one of acrylamide, sodium acryloyldimethyl taurate copolymer, isohexadecane, and polysorbate 80; about 0.6%-1% (wt/wt) of at least one of a jojoba ester, a hydrogenated jojoba oil, and tocopherol acetate; up to about 0.06% (wt/wt) triethanolamine; a fragrance; and a preservative.

20. The composition of claim 13, wherein said emollient comprises at least one of a carrier particle and glycerin.

21. The composition of claim 13, further comprising an additive selected from the group consisting of: a coloring agent, a dye, a color-shifting pigment, a preservative, a pH modifier, a weak base, a pH buffering agent, a thickening agent, a polymer, a fragrance, a polar extract of a fragrance, water, a polyacrylic acid, a sugar alcohol, glitter, a special effect pigment, a vitamin, a pro-vitamin, an amino acid, a protein, a peptide, a peptide complex, and an active agent.

22. The composition of claim 13, wherein said composition is formulated into at least one of a soap, a body wash, a stringent, a toner, a freshener, a gel, a towelette, a napkin, a feminine hygiene product, and a wipe.

23. The composition of claim 13, wherein said composition provides at least one of: improvement of the function of the stratum corneum, reduction in transepidermal water loss, improved substantive feel, and improved moisturizing feel.

24. A method for managing dermatitic symptoms of mammalian integument, said method comprising the step of providing a moisturizing and sanitizing composition for topical application to the skin of a mammalian subject, said composition comprising:
an anti-microbial sanitizer having between about 60%-95% alcohol; and
an emollient comprising at least one of jojoba oil, a jojoba ester, hydrogenated jojoba oil, a jojoba hydrolysate, a hydrolyzed jojoba ester, a jojoba alcohol, an alkoxylated jojoba wax, an alkoxylated and at least partially hydrogenated jojoba wax, an alkoxylated product of jojoba oil interesterified with hydrogenated jojoba oil, and an isopropyl jojobate.

25. The method of claim 24, wherein said moisturizing and sanitizing composition further comprises glycerin.

26. The method of claim 24, wherein said dermatitic symptom comprises at least one of: erythema, scaling, fissuring, xerosis, edema, vesiculation and lichenification.

27. The method of claim 24, wherein said composition provides at least one of: improvement of the function of the stratum corneum, reduction in transepidermal water loss, improved substantive feel, and improved moisturizing feel.

28. A moisturizing hand sanitizer composition, said composition comprising an anti-microbial sanitizer and an emollient selected from the group consisting of jojoba oil, a jojoba ester, hydrogenated jojoba oil, a jojoba hydrolysate, a hydrolyzed jojoba ester, a jojoba alcohol, an alkoxylated jojoba wax, an alkoxylated and at least partially hydrogenated jojoba wax, an alkoxylated product of jojoba oil interesterified with hydrogenated jojoba oil, and an isopropyl jojobate.

29. The composition of claim 28, wherein said moisturizing hand sanitizer composition is certified as organic.
